(19) United States
(12) Patent Application Publication
(10) Pub. No.: US 2004/0067213 A1
(43) Pub. Date: Apr. 8, 2004

(54) Cosmetic and/or pharmaceutical agents

(76) Inventors: Karl Heinz Schmid, Mettmann (DE); Hans-Christian Raths, Monheim (DE); Anke Eggers, Duesseldorf (DE); Ingomar Mrozek, Duesseldorf (DE)

Correspondence Address: COGNIS CORPORATION, PATENT DEPARTMENT, 300 BROOKSIDE AVENUE, AMBLER, PA, 19002, US

(21) Appl. No.: 10/466,618

(22) PCT Filed: Jan. 9, 2002
(86) PCT No.: PCT/EP02/00124

(30) Foreign Application Priority Data
Jan. 18, 2001 (DE) 101 02 009.0

(51) Int. Cl. A61K 7/075; A61K 7/08; A61K 38/00

(52) U.S. Cl. 424/70.22; 514/2

(57) ABSTRACT

Preparations comprising at least one acylated amino acid in an amount of from 40 to 80% by weight and at least one protein condensate in an amount of from 20 to 60% by weight, based on the total weight of the combined at least one acylated amino acid and the at least one protein condensate, are described. Cosmetic and/or pharmaceutical compositions containing said preparations are also described along with methods of preparing emulsions, producing foam and stabilizing foam produced by surfactant compositions.

### FIELD OF THE INVENTION

This invention relates to a preparation of at least one acylated amino acid and at least one protein condensate and to its use as an emulsifier and/or foaming agent.

### PRIOR ART

Besides surfactants, collagen-based protein condensates are added to many cosmetic preparations, for example hair shampoos and body cleansing preparations. Protein condensates such as these show good foaming and cleansing properties and can be inexpensively produced. In view of the ongoing BSE problems, however, protein condensates obtained from animal proteins are no longer used in cosmetic preparations.

Now, the problem addressed by the present invention was to provide protein condensates, optionally in admixture with surfactants, which are obtained from marine and vegetable proteins and from silk, cashmere and milk and which would have excellent foaming and cleansing properties and, in addition, would be suitable for use as emulsifiers.

### DESCRIPTION OF THE INVENTION

The present invention relates to a cosmetic and/or pharmaceutical preparation containing

(a) 40 to 80, preferably 45 to 60 and more particularly 50% by weight of at least one acylated amino acid and

(b) 60 to 20, preferably 55 to 40 and more particularly 50% by weight of at least one protein condensate,

with the proviso that the quantities shown add up to 100% by weight, optionally with water.

The present invention also relates to the use of the preparations according to the invention as emulsifiers and foaming agents.

It has surprisingly been found that mixtures containing acylated amino acids and protein condensates, preferably based on wheat, soya, silk, milk, algae and the like, with a defined composition—in contrast to acylated amino acids and protein condensates on their own—have excellent foam properties (inter alia stable foams). Accordingly, the preparations according to the invention may also be used as foam regulators in surface-active preparations. In addition, the surfactant mixtures according to the invention show excellent emulsifying properties.

Acylated Amino Acids

Acylated amino acids in the context of the invention are any compounds which are obtainable by acylation of amino acids with fatty acid halides corresponding to formula (I):

R¹COX (I)

in which R¹ is an alkyl or alkenyl group containing 6 to 22, preferably 8 to 18 and more particularly 12 to 16 carbon atoms and X is chlorine, bromine or iodine, preferably chlorine, by any of the standard methods known from the prior art. Typical acid halides are octanoyl chloride, nonanoyl chloride, decanoyl chloride, undecanoyl chloride, lauroyl chloride, tridecanoyl chloride, myristyl chloride, palmitoyl chloride, stearoyl chloride, oleoyl chloride and mixtures thereof. The fatty acid halides are used in a molar ratio of acylatable compound to acid halide of 1 to 1.5 and preferably 1.1 to 1.3% by weight for the production of the surfactant mixtures according to the invention. The acylated amino acids thus produced have a degree of acylation of at least 60, preferably 70 and more particularly 85%.

Preferred acylated amino acids are obtained by reaction of glutamic acid, sarcosine, aspartic acid, alanine, valine, leucine, isoleucine, proline, hydroxyproline, lysine, glycine, serine, cystein, cystine, threonine, histidine and salts thereof and, more particularly, glutamic acid, sarcosine, aspartic acid, lysine, glycine and the monosodium salts thereof in optically pure form or as racemic mixtures with fatty acid halides corresponding to formula (I). In one particular embodiment of the invention, cocoacyl glutamate is used.

The amino acids or their salts are used in the surfactant mixture according to the invention in quantities of 40 to 80, preferably 45 to 60 and more particularly 50% by weight, based on the active substance content of the composition as a whole.

Protein Condensates

Protein condensates in the context of the invention are any compounds obtainable by acylation of protein hydrolyzates with fatty acid halides corresponding to formula (I)—as described under the heading of acylated amino acids—by standard methods known from the prior art. According to the invention, the protein condensates have degrees of acylation of 40 to 99, preferably 55 to 93 and more particularly 60 to 85% by weight, based on the active substance content. These degrees of acylation are determined on the basis of the difference between the fatty acid used and the free fatty acid.

Protein hydrolyzates in the context of the invention are degradation products of vegetable proteins, for example wheat, rice, soya, sunflower, almond and potato protein; marine proteins, for example algal protein or protein from marine animals; and milk, silk and cashmere proteins, preferably wheat, rice, soya, sunflower, almond and potato protein and, more particularly, wheat, rice, soya, sunflower, almond and potato protein, which are obtained by acidic, alkaline and/or enzymatic hydrolysis and thereafter have an average molecular weight of 100 to 4,000, preferably 300 to 2,500 and more particularly 400 to 1,200. Although protein hydrolyzates are not surfactants they can be converted into protein condensates which do have surfactant properties by acylation with fatty acid halides. Synthetically obtainable oligopeptides also fall within this claim.

Overviews of the production and use of protein hydrolyzates have been published, for example, by G. Schuster and A. Domsch in Seifen, Öle, Fette, Wachse, 108, 177 (1982) and Cosm. Toil. 99, 63 (1984), by H. W. Steisslinger in Parf. Kosm. 72, 556 (1991) and by F. Aurich et al. in Tens. Surf. Det. 29, 389 (1992). Vegetable protein hydrolyzates based on wheat gluten, soya or rice protein, of which the production is described in German patents DE 19502167 C1 and DE 19502168 C1, are preferably used. One particular embodiment of the invention is characterized by the use of, for example, acylation products of wheat proteins with an average molecular weight of 400 to 1,400, preferably 800 to 1,200, with fatty acids corresponding to formula (I) and preferably coconut fatty acid with a C₈₋₁₈ fatty acid residue.

The protein hydrolyzates are used in the surfactant mixture according to the invention in quantities of 60 to 20, preferably 55 to 40 and more particularly 50% by weight, based on the active substance content of the composition as a whole.

Alcohols

In one particular embodiment of the invention, 0 to 15, preferably 0.2 to 10 and more particularly 0.5 to 6% by weight of mono- or polyhydric alcohols, for example ethanol, propanol, isopropanol, butanol, sec.butanol, methoxypropanol, tert.butanol, glycerol, ethylene glycol, propylene glycol, dipropylene glycol, 1,3-butylene glyol, butane-1,2-diol, butane-1,4-diol, sorbitol, mannitol, erythritol, pentaerythritol, may be added as an additional component.

Production of Acylated Amino Acids and Protein Condensates

The acylated amino acids are produced by the methods known from the chemical literature. The reaction may also be carried out using solvents, such as ethanol, isopropanol, propylene glycol, etc.

Commercial Applications

The preparations according to the invention may be diluted with water to any concentration. The water content may be from 10 to 80% by weight and is preferably from 30 to 70% by weight and more particularly from 40 to 60% by weight.

They may be used in surface-active preparations in quantities of 0.1 to 40, preferably 0.5 to 25 and more particularly 2 to 10% by weight, based on the active substance content.

Surface-active preparations in the context of the invention are, preferably, laundry and dishwashing detergents, cleaners and also cosmetic and/or pharmaceutical preparations, more particularly cosmetic and/or pharmaceutical preparations. These surface-active preparations may contain pearlizing waxes, consistency factors, thickeners, superfatting agents, stabilizers, silicone compounds, fats, waxes, lecithins, phospholipids, antioxidants, deodorants, antiperspirants, antidandruff agents, swelling agents, tyrosine inhibitors, hydrotropes, solubilizers, preservatives, perfume oils, dyes, surfactants and other typical ingredients encountered, for example, in laundry detergents, dishwashing detergents and cleaning compositions as further auxiliaries and additives. Preferred cosmetic and/or pharmaceutical preparations are oral hygiene and dental care preparations, hair shampoos, hair lotions, foam baths, shower baths, creams, gels, lotions, alcoholic and aqueous/alcoholic solutions and emulsions.

The mixtures according to the invention may advantageously be used as foaming agents or as emulsifiers in the surface-active preparations.

Typical cosmetic and/or pharmaceutical cleansing preparations preferably have the following composition (based on the active substance content):

(a) 0.1 to 15, preferably 0.5 to 10 and more particularly 2.0 to 7.5% by weight of the mixture according to the invention of at least one acylated amino acid and at least one protein condensate

(b) 0.05 to 15, preferably 0.5 to 10 and more particularly 2.5 to 7.5% by weight of betaines and optionally

(c) 0 to 15, preferably 0.5 to 10 and more particularly 2.5 to 7.5% by weight of other anionic surfactants,

with the proviso that the quantities shown add up to 100% by weight, optionally with other auxiliaries and additives.

Typical liquid laundry and dishwashing detergents and cleaners preferably have the following composition (based on the active substance content):

(a) 2 to 30, preferably 7 to 25 and more particularly 10 to 20% by weight of the mixture according to the invention of at least one acylated amino acid and at least one protein condensate

(b) 0.05 to 15, preferably 0.5 to 10 and more particularly 2.5 to 7.5% by weight of betaines and optionally

(c) 2.5 to 30, preferably 7 to 25 and more particularly 10 to 20% by weight of other anionic surfactants,

with the proviso that the quantities shown add up to 100% by weight, optionally with other auxiliaries and additives.

Typical cosmetic and/or pharmaceutical emulsions preferably have the following composition (based on the active substance content):

(a) 0.1 to 15, preferably 0.5 to 10 and more particularly 1 to 5% by weight of the mixture according to the invention of at least one acylated amino acid and at least one protein condensate

(b) 3 to 30, preferably 5 to 20 and more particularly 7 to 15% by weight of oil components and optionally

(c) 0.5 to 20 and preferably 2.5 to 10% by weight of consistency factors,

with the proviso that the quantities shown add up to 100% by weight, optionally with other auxiliaries and additives.

The surfactant mixture according to the invention may be used as a foaming agent or as an emulsifier in surface-active preparations such as, for example, laundry and dishwashing detergents, household cleaners, fire extinguishing foams, foam carpets for aircraft and cosmetic and/or pharmaceutical preparations. These surface-active preparations may contain pearlizing waxes, consistency factors, thickeners, superfatting agents, stabilizers, silicone compounds, fats, waxes, lecithins, phospholipids, antioxidants, deodorants, antiperspirants, antidandruff agents, swelling agents, tyrosine inhibitors, hydrotropes, solubilizers, preservatives, perfume oils, dyes, other surfactants and the like as further auxiliaries and additives. Cosmetic and/or pharmaceutical preparations include, for example, oral hygiene and dental care preparations, hair shampoos, hair lotions, foam baths, shower baths, creams, gels, lotions, alcoholic and aqueous/alcoholic solutions and emulsions.

Waxes

Suitable waxes are inter alia natural waxes such as, for example, candelilla wax, carnauba wax, Japan wax, espartograss wax, cork wax, guaruma wax, rice germ oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs) and correspond to the following general formula:

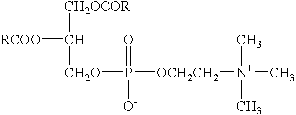

where R typically represents linear aliphatic hydrocarbon radicals containing 15 to 17 carbon atoms and up to 4 cis-double bonds. Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycero-phosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially coconut fatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Antioxidants

Antioxidants which interrupt the photochemical reaction chain that is initiated when UV rays penetrate into the skin may also be added. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Preservatives

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Perfume Oils

Suitable perfume oils are mixtures of natural and synthetic fragrances. Natural fragrances include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Dyes

Suitable dyes are any of the substances suitable and approved for cosmetic purposes as listed, for example, in the publication “Kosmetische Firbemittel” of the Farbstoffkommission der Deutschen Forschungsgemeinschaft, Verlag Chemie, Weinheim, 1984, pages 81 to 106. These dyes are normally used in concentrations of 0.001 to 0.1% by weight, based on the mixture as a whole.

The total percentage content of auxiliaries and additives may be from 1 to 80% by weight and is preferably from 5 to 50% by weight and more particularly from 7 to 10% by weight, based on the particular preparation. The preparations may be produced by standard cold or hot emulsification processes and are preferably produced by the phase inversion temperature (PIT) method.

### EXAMPLES

A1 Sodium N—C₈-C₁₆-Cocoyl Glutamate sodium salt of an N-coconut-C₆₋₁₆-acyl glutamate

A2 Sodium N—C₈-C₁₆-Cocoyl Asparaginate sodium salt of an N-coconut-C₆₋₁₆-asparaginate

A3 Sodium N-Palmitoyl/stearoyl-L-Glutamate sodium salt of an N-coconut-C₁₆₋₁₈-acyl glutamate

A4 Sodium N—C₁₂-C₁₆-Cocoyl-L-Glutamate sodium salt of an N-coconut-C₁₂₋₁₆-acyl glutamate

B1 Sodium N—C₁₂-C₁₈-Cocoyl Hydrolyzed Wheat Protein sodium salt of an acylation product of wheat protein (average molecular weight 1200) with C₁₂₋₁₈ coconut fatty acid

B2 Sodium N—C₁₂-C₁₈-Cocoyl Hydrolyzed Wheat Protein sodium salt of an acylation product of wheat protein (average molecular weight 1200) with C_{12/18} coconut fatty acid

B3 Sodium N—C₁₂-C₁₈-Cocoyl Hydrolyzed Wheat Protein sodium salt of an acylation product of wheat protein (average molecular weight 1200) with C_{12/18} coconut fatty acid

B4 Sodium-N-Palmitoyl/stearoyl Hydrolyzed Wheat Protein sodium salt of an acylation product of wheat protein (average molecular weight 1200) with C_{16/18} coconut fatty acid

TABLE 1
Characteristics of the protein condensates - quantities in % by weight

| Characteristic | B1 | B2 | B3 | B4 |
|---|---|---|---|---|
| Active substance content* | 39.8 | 41 | 30.6 | 39 |
| Total nitrogen | 3.23 | 3.48 | 3.01 | 3.32 |
| Free fatty acid | 4.3 | 2.9 | 1.8 | 2.1 |
| Degree of acylation** | 93 | 56 | 45 | 62 |

*active substance = dry residue
**calculated from the difference (fatty acid used - free fatty acid)

The surfactant mixtures according to the invention (Examples 1 to 5), comparison surfactant mixtures (C5 and C6) and the individual components (Comparison Examples C1 to C4) were tested for their foaming capacity in hard water. To this end, the “beaten” foam volume (ml) was determined at different times (30 seconds, 5, 10, 15 and 20 minutes) by Götte's beaten foam method (DIN 53902, 3/81; 15° dH; pH 6; 40° C.].

TABLE 2

| | | | | | | | | | | | |
|---|---|---|---|---|---|---|---|---|---|---|---|
| Composition | 1 | 2 | 3 | 4 | 5 | C1 | C2 | C3 | C4 | C5 | C6 |

Foaming capacity in g/l active substance

| | | | | | | | | | | | |
|---|---|---|---|---|---|---|---|---|---|---|---|
| A1 | 1.0 | — | 1.0 | 0.5 | 1.2 | 2.0 | | — | — | 1.8 | 0.4 |
| A2 | — | 1.0 | — | 0.5 | — | — | | — | — | — | — |
| B1 | 1.0 | — | — | — | 0.8 | — | 2.0 | — | — | 0.2 | 1.6 |
| B2 | — | 1.0 | — | — | — | — | — | 2.0 | — | — | — |
| B3 | — | — | 1.0 | 1.0 | — | — | — | — | 2.0 | | |

Foam volume [ml]

| | | | | | | | | | | | |
|---|---|---|---|---|---|---|---|---|---|---|---|
| After 30 s | 350 | 275 | 250 | 240 | 400 | 350 | 130 | 80 | 60 | 300 | 100 |
| After 5 mins. | 290 | 270 | 240 | 230 | 350 | 0 | 100 | 70 | 50 | 50 | 90 |
| After 10 mins. | 285 | 265 | 235 | 220 | 330 | 0 | 70 | 55 | 40 | 20 | 90 |
| After 15 mins. | 280 | 260 | 230 | 210 | 310 | 0 | 60 | 45 | 30 | 10 | 80 |
| After 20 mins. | 275 | 255 | 220 | 200 | 290 | 0 | 45 | 20 | 15 | 0 | 70 |

O/W creams were prepared using the combinations according to the invention of N-acyl glutamates and N-acyl protein condensates (Table 3, Examples 1 to 5) and of the respective individual components or comparison surfactant mixtures (Table 3, Comparison Examples C1 to C7). The creams were stored for a total of 12 weeks at 20, 40 and 45° C. and the stability of the emulsions was evaluated [(+)=stable and (−)=separation].

TABLE 3
Emulsifying capacity - quantities based on % by weight active substance

| | | | | | | | | | | | |
|---|---|---|---|---|---|---|---|---|---|---|---|
| 1 | 2 | 3 | 4 | 5 | C1 | C2 | C3 | C4 | C5 | C6 | C7 |

Components

| A3 | 1.0 | 1.0 | — | 0.5 | 1.2 | 2.0 | 2.0 | — | — | — | 1.4 | 0.6 |
|---|---|---|---|---|---|---|---|---|---|---|---|---|
| A4 | — | — | 1.0 | 0.5 | — | — | — | 2.0 | — | — | — | — |
| B4 | 1.0 | 1.0 | — | — | 0.8 | — | — | — | 2.0 | — | 0.6 | 1.4 |
| B1 | — | — | 1.0 | 1.0 | — | — | — | — | — | 2.0 | — | — |
| Cetearyl alcohol | 7.0 | 7.0 | 7.0 | 7.0 | 7.0 | 7.0 | 7.0 | 7.0 | 7.0 | 7.0 | 7.0 | 7.0 |
| Paraffin oil | 16.0 | — | — | — | — | 16.0 | — | — | — | — | — | — |
| Dicaprylyl Ether | — | 4.0 | 4.0 | 4.0 | 4.0 | — | 4.0 | 4.0 | 4.0 | 4.0 | 4.0 | 4.0 |
| Coco-Caprylate/Caprate | — | 7.0 | 7.0 | 7.0 | 7.0 | — | 7.0 | 7.0 | 7.0 | 7.0 | 7.0 | 7.0 |
| Capric/Caprylic Triglyceride | — | 5.0 | 5.0 | 5.0 | 5.0 | — | 5.0 | 5.0 | 5.0 | 5.0 | 5.0 | 5.0 |
| Water | to 100 | | | | | | | | | | | |
| Stability | | | | | | | | | | | | |
| 1 week, 20° C. | + | + | + | + | + | + | + | + | + | + | + | + |
| 4 weeks, 20° C. | + | + | + | + | + | + | + | + | + | + | + | + |
| 12 weeks, 20° C. | + | + | + | + | + | + | + | + | + | + | + | + |
| 1 week, 40° C. | + | + | + | + | + | + | + | − | + | + | + | + |
| 4 weeks, 40° C. | + | + | + | + | + | + | + | − | + | + | + | + |
| 12 weeks, 40° C. | + | + | − | + | + | + | + | − | + | − | + | + |
| 1 week, 45° C. | + | + | − | − | + | + | + | − | + | − | + | + |
| 4 weeks, 45° C. | + | + | − | − | + | − | + | − | − | − | + | − |
| 12 weeks, 45° C. | + | + | − | − | + | − | − | − | − | − | − | − |

1. Cosmetic and/or pharmaceutical preparation containing
(a) 40 to 80% by weight of at least one acylated amino acid and
(b) 60 to 20% by weight of at least one protein condensate,
with the proviso that the quantities shown add up to 100% by weight, optionally with water.

2. Preparation as claimed in claim 1, characterized in that
(a) 45 to 60% by weight of at least one acylated amino acid and
(b) 55 to 40% by weight of at least one protein condensate,
with the proviso that the quantities shown add up to 100% by weight, optionally with water, are used.

3. Preparation as claimed in claims 1 and/or 2, characterized in that acylated amino acids obtained by reaction of amino acids with fatty acid halides corresponding to formula (I):
R¹COX (I)
in which R¹ is an alkyl or alkenyl group containing 6 to 22 carbon atoms and X is chlorine, bromine or iodine,
are used.

4. Preparation as claimed in at least one of claims 1 to 3, characterized in that acylated amino acids formed by reaction of glutamic acid, sarcosine, aspartic acid, alanine, valine, leucine, isoleucine, proline, hydroxyproline, lysine, glycine, serine, cysteine, cystine, threonine, histidine and salts thereof with fatty acid halides corresponding to formula (I) are used.

5. Preparation as claimed in at least one of claims 1 to 4, characterized in that protein condensates formed by reaction of protein hydrolyzates based on vegetable or marine proteins and on milk, silk or cashmere proteins with fatty acid halides corresponding to formula (I) are used.

6. Preparation as claimed in at least one of claims 1 to 5, characterized in that acylated amino acids with a degree of acylation of at least 70% and protein condensates with a degree of acylation of at least 40% are used.

7. Preparation as claimed in at least one of claims 1 to 6, characterized in that protein condensates with an average molecular weight of 100 to 4,000 are used.

8. Cosmetic and/or pharmaceutical compositions containing 0.1 to 40% by weight of the preparation claimed in at least one of claims 1 to 7.

9. The use of the surfactant mixture claimed in claim 1 as a foaming agent.

10. The use of the surfactant mixture claimed in claim 1 as an emulsifier.
